(19) United States
(12) Patent Application Publication
(10) Pub. No.: US 2019/0216657 A1
(43) Pub. Date: Jul. 18, 2019

(54) Diaper to Absorb Urine for Incontinence Males

(71) Applicant: Louis Thomas, Ontario, CA (US)

(72) Inventor: Louis Thomas, Ontario, CA (US)

(21) Appl. No.: 15/870,664

(22) Filed: Jan. 12, 2018

(51) Int. Cl. A61F 13/471; A61F 13/47

(52) U.S. Cl. CPC A61F 13/471; A61F 2013/15121; A61F 13/4704

(57) ABSTRACT

The present disclosure relates to an incontinence diaper for use by males. The diaper encloses a penis that is secured to the diaper by a strap found within the diaper. This diaper improves the prior art by minimizing the amount of skin that is exposed to urine that is involuntarily escaping the penis, accommodate penises of varying diameter and lengths, may be reused, and is simple to use and discreet.

### BACKGROUND OF THE INVENTION

### 1. Field of the Invention

This invention to the general field of incontinence products. In particular, it provides a new and improved diaper for incontinent males who, because of disabilities, are unable to care for themselves and require assistance for their hygienic needs.

### 2. Description of the Related Art

Urinary incontinence among males is not an unusual common condition that results from a number of different causes. This condition is seen as highly distressing due to the fact that accidental leakage occurs without knowledge of male suffering from the condition and when occurring in the presence of others can lead to high levels of embarrassment, unwanted odors, additional washing of affected clothing, and skin irritations and rashes. Due to the uncontrollable nature of this condition, constant precautions must be taken in order to prevent accidental mishaps at inopportune times.

While accidental leakages are viewed as highly embarrassing, many conventional protective measures can lead to embarrassment in and of themselves. For instance, absorbent incontinence pads are often bulky and can be clearly seen in outline form under one's clothes. Additionally, they are uncomfortable and hot to wear, especially in warm weather. In general, the amount of protection provided is inversely proportional to the discomfort and visibility of the protecting device.

By way of example, U.S. Pat. No. 5,036,839 was issued in Aug. 6, 1991 to Arieh Weiss and Raymond M. Weiss. She discloses a supporter that encloses the scrotum and penis which contains an aperture that allows the penis to pass through. Her supporter is a chamber for the testes which includes an opening for the penis and is secured by a strap encircling both penis and testes. The support is difficult to secure as the strap must go behind the scrotum where it is not visible to the user and exposes the skin of the scrotum to urine excreting from the penis causing skin irritations and rashes on the scrotum.

U.S. Pat. No. 5,074,853, issued on Dec. 24, 2007 to Bryant; Tracy L. describes a diaper that is wrapped around the penis of a user only, so that adjacent body parts are not in contact with the inside portion of the diaper. The diaper permits a nurse to single-handedly slide it in place around the penis while the patient is either sitting or lying on his back without any need for lifting or moving him It is also shaped to provide a compact fit for comfort and tight adherence to the body to avoid the spreading of wetness during urination. This design improves on the design by Weiss in that only the penis was exposed to possible contact with urine as opposed to both the penis and the scrotum. However Bryant's design did not provide a secure means to attach the penis to the diaper thus resulting in Bryant's diaper becoming misplaced as the user moves about, particularly if the user is rather active.

Also in the prior art are pouches or bags for male incontinence in which the penis of the user in inserted into the pouch through a hole in the wall thereof as described in U.S. Pat. No. 6,569,135 to Mula. However, the dimensions of the hole can not be chosen so that all penis sizes will have a close fit in the hole. If the hole is too small irritation may develop about the shaft of the penis that is in continuous contact with the diaper. If the hole is too large the diaper may come loose from the penis rendering it ineffective and causing embarrassment to the user.

U.S. Pat. No. 6,635,038 to Scovel discloses an incontinence device having a disposable urinary bag having a plurality of attachment means located on its outside surface for securement of the disposable urinary bag to the adjustable attachment belt. The user inserts his penis through a hole present on the disposable urinary bag but the disposable urinary bag is secured to the user though the adjustable attachment belt. With the Scovel design even an active wearer of his diaper will feel confident that the diaper will not move from its position because of the adjustable attachment belt encircling the user. In addition, the penis is totally encapsulated limiting any skin exposure to urine. However, the Scovel design requires that the user wear the adjustable attachment belt which is time consuming to fit and adds to the complexity of the Scovel design.

Known devices in the art do not provide a one piece solution that may be securely attached to the penis. It would be very desirable to those males affected by incontinence to have access to a diaper that limits the amount of skin exposed to urine to the greatest degree possible while providing a secure fit with the penis so as to give the wearer confidence that the diaper will not slip off of the penis.

### BRIEF SUMMARY OF THE INVENTION

A urine absorbent pouch for male incontinence is provided. The urine absorbent pouch may be of the same design as typical diapers for babies and is generally in a rectangular shape where its length is greater than its width. A fold line exists bisecting at the midpoint of its longitudinal axis. The outer surface of the diaper is comprised of a fabric that prevents the passage of liquids while the inner layers are comprised of absorbent materials that will hold and contain any urine that discharges from the penis. At one end of the diaper are two resealable adhesive strips, one at either side of the diaper, that are used to secure the ends of the diaper together when the diaper is folded about the penis. At the same end where the adhesive strips are found, a strap is provided to secure the penis to the diaper. The strap is generally elastic and is of sufficient width so that it may be laid across any size penis in comfort. Additionally, the strap is of such length to accommodate penises with varying diameters. The strap is generally rectangular with a length greater than its width with the ends being defined along its longitudinal axis. The first end is attached to the diaper while the second end is removably attached to the diaper.

To use the diaper the user will first place his penis so that it rests perpendicular to the strap. The strap is then laid across and over the top of the penis and the end of the strap is removably attached to the diaper. Finally the user will fold the diaper along the fold line and secure the two ends of the diaper together using the two resealable adhesive strips. At this point the penis is secured to the diaper and the penis is totally enclosed by the diaper.

An object of the present invention is to reduce the size of the diaper to the extent possible so that the diaper is not noticed and remains discreet.

It is further an object of the present invention is to limit exposure to urine to the user's penis only so that skin about the penis remains dry.

It is further an object of the present invention to accommodate penises of varying thicknesses and lengths with a single design.

It is further an object of the present invention to allow the user of the diaper to relieve themselves without much more complications than a male not affected by incontinence and if needed to allow the diaper to be reused after relieving themselves.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

Neither this summary nor the following detailed description defines or limits the invention. The invention is defined by the claims.

### BRIEF DESCRIPTION OF DRAWINGS

The present invention will become more fully understood from the detailed description and accompanying drawings, wherein:

FIG. 1 shows the diaper of the present invention with a penis laid therein with the strap in position to secure the penis to the diaper.

FIG. 2 shows the diaper of the present invention with a penis laid therein and secured to the diaper with the strap.

### DETAILED DESCRIPTION OF THE INVENTION

The following discussion describes in detail one embodiment of the invention. This discussion should not be construed, however, as limiting the invention to this particular embodiment. Practitioners skilled in the art will recognize numerous other embodiments as well. For a definition of the complete scope of the invention, the reader is directed to the appended claims.

The present invention describes a diaper 10 for use by males with incontinence which provides a means for preventing discomfort, accidents, and embarrassment for males suffering from incontinence.

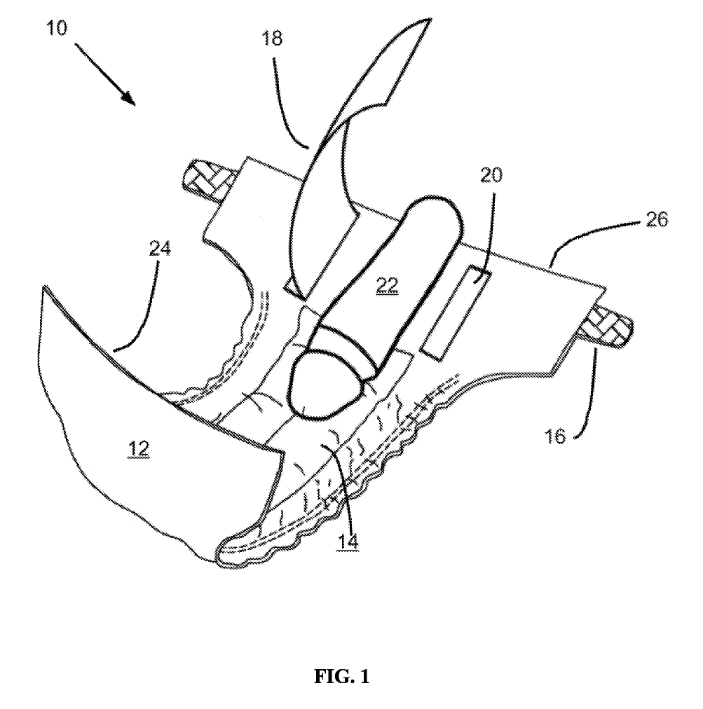

Turning to FIG. 1 a perspective view of diaper 10 is shown with penis 22 laid thereon with strap 18 in its open position as it would be prior to securing penis 22 to diaper 10. Diaper 10 is generally rectangular in shape with a length greater than its width and two ends, front 24 and back 26, along its longitudinal axis. A fold line exists but is not shown in FIG. 1 at the midpoint of diaper 10 longitudinal axis so as to facilitate the bringing of front 24 to back 26. Furthermore, diaper 10 has outer fabric 12 that is waterproof and prevents any liquids, particularly urine present in absorbent material 14, from escaping diaper 10. Attached to outer fabric 12 is absorbent material 14, a layer of highly absorbent material designed to readily soak up any urine that involuntarily escapes from penis 22. Reusable adhesive strips 16, present at either end of back 26, serves to secure back 26 with front 24 when diaper 10 is folded over penis 22. When front 24 is folded to back 26 strips 16 engage outer fabric 12 present at front 26 of diaper 10 to secure diaper 10 in its folded position. Strips 16 may be peeled away from outer fabric 12 and re reattached to outer fabric 12 on those occasions the user wishes to relieve themselves or for whatever other reason the user wishes to temporarily open diaper 10. Strap 18 is generally rectangular in shape with a length greater than its width and with ends along its longitudinal axis. The first end of strap 18 is attached to diaper 10 while opposing second end may be removably attached to pad 20 via some sort of hook and loop solution such as Velcro or other such solutions that are extensively available in the field.

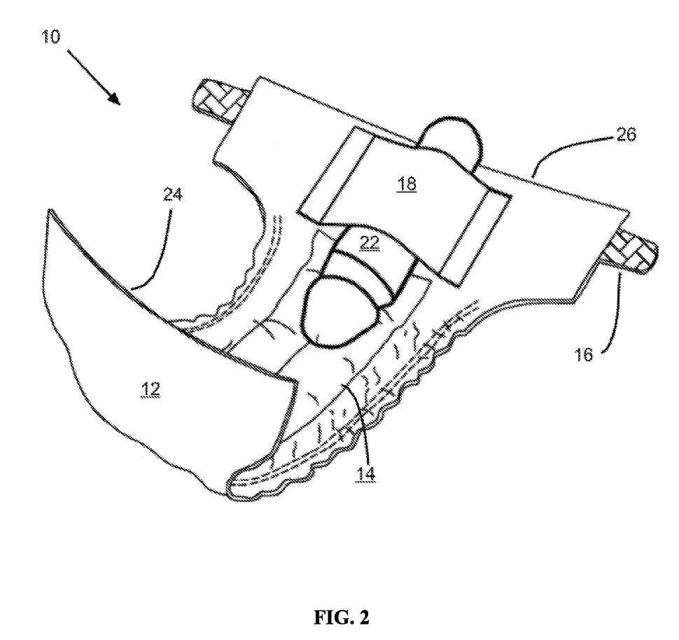

FIG. 2 shows diaper 10 with penis 22 secured to diaper 10 by strap 18. Strap 18 is of such a length so as to accommodate penises of varying diameters and of such a width so as to not cause discomforted when laid across the top of the penis.

To use diaper 10 the user will first lay his penis on top of absorbent material 14 and secure strap 18 over the top of penis 22. The user will then fold diaper 10 along the fold line at the midpoint of diaper 10 longitudinal axis and bring front 24 to back 26 and secure the fold with strips 16.

With respect to the above description then, it is to be realized that the optimum dimensional relationships for the parts of diaper 10 enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by the embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. (canceled)

2. A male incontinence diaper to contain a penis that is generally rectangular of a length greater than its width with proximal end and distal end along its longitudinal axis having:
a waterproof outer fabric;
a liquid absorbing material attached to said outer fabric forming an inner layer;
a strap positioned adjacent to said proximal end that is generally rectangular of a length greater than its width with ends along its longitudinal axis with first end attached to said inner layer and second end being removably engagable with a pad attached to said inner layer said strap securing said penis to said diaper by laying said strap over said penis and engaging said second end with said pad; and
two adhesive means attached to said outer fabric and positioned adjacent said distal end of said diaper to removably engage said proximal end of said diaper when said diaper folds to enclose said penis.
